(19) United States
(12) Patent Application Publication
(10) Pub. No.: US 2002/0146748 A1
(43) Pub. Date: Oct. 10, 2002

(54) Immunological detection of neosporosis using a recombinant antigen

(76) Inventors: L. David Sibley, University City, MO (US); Daniel K. Howe, Lexington, KY (US)

Correspondence Address: Alfred W Zaher, Centre Square West, 1500 Market Street, Philadelphia, PA, 19102-2186, US

(21) Appl. No.: 09/767,037

(22) Filed: Jan. 22, 2001

(51) Int. Cl. G01N 33/53; G01N 33/569; C07H 21/04; C12N 9/00; C07K 16/20

(52) U.S. Cl. 435/7.22; 435/183; 536/23.2; 530/388.2

(57) ABSTRACT

An immunological assay method is disclosed which utilizes recombinant antigen, rNcp29, derived from an immunodominant surface antigen of Neospora caninum tachyzoites. Specifically, an ELISA method is disclosed. The method provides sensitive and specific detection of antibodies in sera of infected animals and does not exhibit cross-reaction with antisera against related parasites such as T. gondii. The ELISA method is used to screen animals for the presence of serum antibodies specific to recombinant Ncp29.

Pursuant to 35 U.S.C. §202(c), it is acknowledged that the U.S. Government has certain rights in the invention described herein, which was made in part with funds from the United States Department Agriculture, Grant No. 97-35204-4770.

### FIELD OF THE INVENTION

This invention relates to the field of disease diagnosis and epidemiology. More specifically the invention relates to the sensitive and specific immunological detection of Neospora caninum infections, using a recombinant antigen.

### BACKGROUND OF THE INVENTION

Various scientific articles and patent documents are referred to throughout the specification to describe the state of the art to which this invention pertains. Each of these documents is incorporated by reference herein in its entirety.

Neospora caninum is a relatively newly recognized, coccidian parasite that causes abortions and neonatal morbidity and mortality in cattle, sheep, goats, and horses. Neosporosis is an emerging, economically-significant disease in cattle where it causes abortion and paralysis of newborn animals (Dubey, J. P. Int'l J. Parasit. 1999; 29: 1485-1488). Outbreaks of neosporosis are sporadic but have been reported in the United States (California, Colorado, South Dakota, Midwestern States), Sweden, The Netherlands, United Kingdom, South Africa, Japan and Australia.

Neosporosis is also an significant cause of paralysis in dogs. It has been discovered recently that the dog is capable of oral-fecal transmission of this parasite. Herbivorous mammals, including dairy cows, presumably become infected by ingesting the resistant oocysts (a spore-like stage) that are passed in dog feces and which contaminate feed, range, and water sources. Thus, control of neosporosis will ultimately rely on detection and treatment of the infection in both the dog and the various intermediate hosts such as cattle.

N. caninum is closely related to Toxoplasma gondii, based on ribosomal small subunit RNA sequences; they further share significant morphological, life-cycle, and similarities at the molecular level. Due to the high degree of similarity between organisms, serological diagnosis is complicated by the potential for false-positives resulting from antigenic cross-reactivity of antibodies to closely-related parasites such as Toxoplasma gondii and Sarcocystis cruzi that also infect cattle.

Evidence of the close similarity among these organisms includes the many T. gondii gene homologs that are found in N. caninium. Recently, for example, close homologs of a nucleoside triphosphate hydrolase, known as NTPase (Asai et al. Exp. Parasitol. 1998; 90: 277-285), as well as a member of a conserved family of adhesins, known as MIC2 (Lovett et al., Molec. Biochem. Parasitol. 2000; 107: 33-43), which is involved in recognition of the host cell, have been characterized. In each case, these proteins are approximately 60-70% identical at the amino acid level and polyclonal antibodies to the T. gondii protein react to the corresponding N. caninum protein.

Research has shown that the major surface antigens of N. caninum, known as Ncp29 (also called Nc-p36) and Ncp35 (also called Nc-p43), belong to a family of antigens that were first identified in T. gondii and are characterized by the conservation of cysteine residues. In T. gondii, these surface antigen genes are collectively known as SAGs. Ncp29 is encoded by a gene that is homologous to the SAG1 gene previously characterized from T. gondii; Ncp35 is a homolog of T. gondii SRS2. In both organisms, these surface antigens tend to be immunodominant antigens. Despite the fact that these genes are related and the proteins they encode are both highly immunogenic, infection with N. caninum elicits an antibody response that is specific to Ncp29 and which is not observed in animals infected with related parasites (Howe et al., Int'l J. Parasit. 1999; 29: 1489-1496).

N. caninum infection in animals is most easily detected by the presence of antibodies to the protozoan in the serum. Bjorkman and Uggla, Int'l J. Parasit. 1999; 29: 1497-1507) have reviewed the serological diagnosis of Neospora caninum infection. A diagnostic immunofluorescent antibody test (IFAT) was first developed for N. caninum using whole parasite antigens from cultures adapted for growth in vitro. While an important development for establishing the etiology of protozoal abortion in cattle, this assay requires experienced, trained personnel and is subjective in nature, thereby making the results difficult to compare among different laboratories. Due to the reliance on whole antigens, the IFAT assay also increases the likelihood of cross-reaction with antibodies to conserved antigens that are found in other related parasites, such as T. gondii.

In addition to IFAT, other immunological assays for the detection of Neospora have been developed. The art has moved to ELISA as a method for detection. Bjorkman et al (Parasite Immunol. 1994; 16: 643-648), Pare et al. (J. Vet. Diagn. Invest. 1995; 7:352-359), and Dubey et al. (J. Parasitol. 1997; 83: 1063-1069) all have proposed variations of ELISA methods for the detection of Neospora caninum. The main goal was to distinguish N. caninum from T. gondii. To this end, those researchers attempted to use monoclonal antibodies that would recognize N. caninum but not T. gondii antigens, or they attempted to find and make recombinant antigens that were specifically recognized by antibodies to N. caninum but not by those to T. gondii.

Isolation of recombinant parasite antigens that react to sera from N. caninum infected animals has been used to identify several candidate antigens. Two such clones, initially called Nc4.1 and Nc14.1, were used in an ELISA assay to detect antibodies in the serum of infected versus non-infected cows (Liddell et al., Mol. Biochem. Parasit. 1998; 93: 153-158; Lally et al. Clin. Diag. Lab. Immunol. 1996; 3: 275-279; Jenkins et al., Clin. Diag. Lab. Immunol. 1997; 4: 270-274). While the assay was reported to be highly specific, the separation in optical density (O.D.) values between positive and negative values was less than 0.1 O.D. unit in some cases, suggesting it may be of limited values in screening samples of unknown status. Completion of the sequences originally identified by Nc14.1 and Nc4.1 revealed they are homologous to the GRA6 and GRA7 genes, respectively, of T. gondii which encode proteins found in secretory dense granules. Other attempts to develop sensitive and specific immunological methods for N. caninum utilized the recombinant antigens N54 and N57 (Louie et al., Clin. Diag. Lab. Immunol. 1997; 4: 692-699). These antigens comprise a subtilysin-like protease (clone N54) and a fragment of the gene called Nc4.1 (clone N57) above by Lally et al. (Mol. Bichem. Parasit. 1997: 87: 239-243. These antigens reportedly perform better than whole parasite extracts in ELISA; however many animals with known exposure still display low titers using these assays (Louie et al., 1997, supra). Sonda et al. (Mol. Biochem. Parasit. 1998; 97: 97-108) also cloned and sequenced the Ncp29 gene and developed a recombinant Neospora antigen that lacked the N-terminal 93 amino acids of the protein. The protein did retain antigenicity sufficient to enable ELISA differentiation of bovine sera from two infected versus two uninfected animals. While this assay format conformed to that typically called an ELISA, rigorous testing was not provided making it impossible to predict if this assay would provide a positive predictive value and low false positive rate on a wider sample of animals with known exposure to N. caninum.

In addition to the above described antigens and methods, U.S. Pat. No. 5,707,617 to Conrad et al. generally discloses biologically pure cultures of Neospora caninum, and further teaches generally the detection of antibodies specifically immunoreactive with a ‘bovine Neospora antigen in a biological sample’. U.S. Pat. No. 5,889,166 to Conrad et al. discloses specific Neospora recombinant antigens and polynucleotides, and the immunological detection of the antigens. U.S. Pat. No. 5,942,394 to Ellis et al. teaches a method of detecting protozoan parasites, particularly N. caninum and T. gondii using PCR-like amplification of nucleic acids present in the sample.

From the foregoing discussion, it is clear that there is a need in the art for a specific, sensitive and accurate assay method for the detection Neospora caninum. Specifically, what is needed is the development of a standardized diagnostic test that offers reliable, sensitive and specific detection based on defined Neospora-specific molecules.

### SUMMARY OF THE INVENTION

The present invention provides an improved immunological assay method for the detection of the parasitic disease, neosporosis. The invention, in a preferred embodiment, comprises an enzyme-linked immunosorbent assay (ELISA) which is provided based on the use of a novel recombinant antigen (sometimes abbreviated herein as Ag). The ELISA assay disclosed herein provides enhanced sensitivity over the methods currently available.

A method for the detection of antibodies to Neospora antigens is also provided in the present invention. The method preferably comprises an ELISA-type assay. The method comprises the steps of providing the aforementioned recombinant antigen; contacting the antigen with an isolated biological fluid under conditions which allow for antibodies present in the biological fluid to bind the recombinant antigen and form Ab-Ag complexes with it; and detecting the Ab-Ag complexes formed after separating the complexes from the unbound Ab and/or Ag, and quantitating the amount of the complexes.

Also provided in accordance with the present invention is a method of assessing an animal's status with respect to Neospora caninum infection. This method is of particular utility in agronomic applications, especially as relates to the sciences of animal husbandry and herd management. The method combines using the detection method of the invention along with known positive and negative controls and optionally, standard curves and other standards, and established assessment criteria to assess the status of each animal in a group. This assessment allows preventive veterinary measures, disease control, proper epidemic containment, culling, and other animal management decisions to be made in an informed manner.

The invention further features the recombinant antigen that yields the improved immunological assay described above, as well as its encoding polynucleotide and other biological molecules related to the antigen. Thus, in accordance with the present invention, an isolated nucleic acid encoding the antigen of the invention is provided. The isolated nucleic acid encodes an antigenic polypeptide, rNcp29, which is the substantially entire mature form of the Neospora caninum protein known as Ncp29.

Also provided in accordance with the present invention is an isolated antigen comprising a particular portion of a major immunodominant Neospora surface protein, Ncp29; this protein is present only during the tachyzoite life-stage of the organism. The Ncp29 protein as it naturally occurs is produced as a protein precursor molecule. The isolated antigen of the present invention, rNcp29, corresponds substantially to the mature portion of the protein. The protein is 269 amino acids long, and differs from the precursor form of Ncp29 in lacking the 30 amino acid signal peptide at its amino terminus and in further missing the four terminal amino acids at its carboxy terminus. In a preferred embodiment, the amino acid sequence of the protein is modified to include a C-terminal six-His tag to facilitate purification of the protein.

Further provided in accordance with the present invention is a recombinant expression system which comprises the isolated nucleic acid encoding the antigen of the invention. The invention provides expression systems based on bacterial or yeast expression. Alternatively the invention also provides expression systems based on other commonly-used organisms such as insect-based expression and/or plant- or animal-based expression systems.

In addition to the recombinant expression systems provided, further in accordance with the present invention is a recombinant antigenic protein product of such a system. The recombinantly-produced antigenic protein of the invention corresponds substantially in its amino acid sequence to that of the mature form of the Ncp29 protein from Neospora caninum. In a preferred embodiment, the antigen is easily purified from the recombinant expression system in which it is made due to the presence of polyHis tags at the C-terminal end of the protein. The invention further provides for antigen purified from recombinant expression systems by rapid techniques such as immobilized metal affinity chromatography.

Further provided in accordance with the present invention are isolated antibodies, or fragments thereof, specific for one or more epitopes of the antigen of the invention. Provided antibodies included polyclonal and monoclonal antibodies. Also provided in accordance with this aspect of the invention are purified or partially purified antibodies, or fragments thereof, which retain immunological specificity for the antigen of the invention. Other antibodies provided by the invention include antibodies specific for the antibodies which bind to the antigen of the invention. Such antibodies are useful in many immunological assays and detection systems.

Other features and advantages of the present invention will be better understood by reference to the drawings, detailed description and examples that follow.

### BRIEF DESCRIPTION OF THE DRAWINGS

FIG. 1: A Western blot analysis of recombinant antigen (lanes labeled rNcp29) and total lysate from N. caninum (lanes labeled Nc-1). The left panel (labeled mAb 6C11) was detected using the mouse monoclonal antibody 6C11, a clone specific for native Ncp29 protein. The right panel (labeled Mouse α:-Nc-1) was detected with mouse polyclonal antiserum generated against N. caninum strain Nc-1.

FIG. 2: ELISA assay of bovine samples, using recombinant antigen, rNcp29. Scatter plot showing the grouping of results of ELISA tests on ten negative (“Negative Herd Samples”); ten positive bovine sera (“Positive Herd Samples”) and Experimental Controls. The experimental controls consisted of three samples each pre-(−) and post-(+) inoculation serum sample from cows experimentally infected with N. caninum.

FIG. 3: ELISA assay of bovine serum samples from infected animals, using recombinant antigen, rNcp29. Scatter plot showing the grouping of results of ELISA tests on bovine sera from animals that were naturally infected with N. caninum (labeled α-N. caninum antisera) or experimentally infected with Toxoplasma gondii (labeled α-T. gondii antisera) or Sarcocystis spp. (labeled α-Sarcocystis antisera). The symbol * indicates a serum sample from an animal previously infected with N. caninum.

### DETAILED DESCRIPTION OF THE INVENTION

Definitions

Various terms relating to the biological molecules of the present invention are used throughout the specifications and claims. Where used herein, “isolated” means altered “by the hand of man” from the natural state. If a composition or substance occurs in nature, it has been “isolated” for example, when changed or removed from its original environment. For example, a unmodified polynucleotide or an unmodified polypeptide naturally present in a living animal is not “isolated,” but the same polynucleotide or polypeptide modified, or separated from the coexisting materials of its natural state, or present through synthetic means, is “isolated”, as the term is employed herein.

With reference to nucleic acids of the invention, the term “isolated nucleic acid” is sometimes used. This term, when applied to genomic DNA, refers to a DNA molecule that is separated from sequences with which it is immediately contiguous (in the 5′ and 3′ directions) in the naturally-occurring genome of the organism from which it was derived. For example, the “isolated nucleic acid” may comprise a DNA molecule inserted into a vector, such as a plasmid or virus vector, or integrated into the genomic DNA of a prokaryote or eukaryote. An “isolated nucleic acid molecule” may also comprise a cDNA molecule or a synthesized nucleic acid molecule. An “isolated nucleic acid” also may be a synthetic nucleic acid.

With respect to RNA molecules of the invention the term “isolated nucleic acid” primarily refers to an RNA molecule encoded by an isolated DNA molecule as defined above. Alternatively, the term may refer to an RNA molecule that has been sufficiently separated from RNA molecules with which it would be associated in its natural state (i.e., in cells or tissues), such that it exists in a “substantially pure” form (the tenn “substantially pure” is defined below). Alternatively, an entire class of RNA molecules is sometimes deemed “isolated” when is separated from other biomolecules and/or other classes of RNA (e.g. tRNA and rRNA). For example, the class of polyadenylated RNA is often isolated in order to clone cDNA from a specific messenger RNA.

With respect to protein, the term “isolated protein” or “isolated and purified protein” is sometimes used herein. This term often refers to a protein which has been sufficiently separated from other proteins with which it would naturally be associated, so as to exist in “substantially pure” form. Alternatively, this term may refer to a protein produced by expression of an isolated nucleic acid molecule of the invention.

An “isolated protein” also may be a synthetic polypeptide comprising naturally occurring or non-naturally occurring amino acid residues.

The terms “nucleic acid” and “polynucleotide” generally refers to any polyribonucleotide or polydeoxribonucleotide, which may be unmodified RNA or DNA or modified RNA or DNA. “Polynucleotides” include, without limitation, single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term “polynucleotide” also includes DNAs or RNAs containing one or more modified bases and DNAs or RNAs with backbones modified for stability or for other reasons. “Modified” bases include, for example, tritylated bases and unusual bases such as inosine. A variety of modifications have been made to DNA and RNA; thus, “polynucleotide” embraces chemically, enzymatically or metabolically modified forms of polynucleotides as synthesized or as typically found in nature, as well as the chemical forms of DNA and RNA characteristic of viruses and cells. “Polynucleotide” also encompasses relatively short polynucleotides, often referred to as oligonucleotides. Such oligonucleotides could be isolated from nature or more typically, chemically synthesized.

The term “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 amino acids represented by codons in the genetic code. “Polypeptides” include amino acid sequences modified either by natural processes, such as post-translational modification or processing, or by chemical modification techniques which are well known in the art. Such modifications are described in basic texts and in more detailed monographs, as well as in extensive research literature. Modifications can occur anywhere in a polypeptide, including the peptide backbone, the amino acid side-chains and the amino and/or carboxyl termini. It will be appreciated that the same type of modification may be present to the same extent or to varied extents at several sites in a given polypeptide. Also, a given polypeptide may contain many types of modifications. Polypeptides may be branched as a result of ubiquitination, and they may be cyclic, with or without branching. Disulfide bridges may form within or between polypeptide chains. Cyclic, branched and branched cyclic polypeptides may result from natural post-translational processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent cross-links, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, PROTEINS—STRUCTURE AND MOLECULAR PROPERTIES, 2nd Ed., T. E. Creighton, W. H. Freeman and Company, New York, 1993 and Wold, F., Posttranslational Protein Modifications: Perspectives and Prospects, pgs. 1-12 in POSTTRANSLATIONAL COVALENT MODIFICATION OF PROTEINS, B. C. Johnson, Ed., Academic Press, New York, 1983; Seifter et al., “Analysis for protein modifications and nonprotein cofactors”, Meth Enzymol (1990) 182:626-646 and Rattan et al., “Protein Synthesis: Posttranslational Modifications and Aging”, Ann NYAcad Sci (1992) 663:48-62. In addition to these modifications and alterations of polypeptides, proteins may also associate with each other in various ways. Where used herein, “dimers” are an association of two proteins to form a single functional unit. “Homodimers” contain two identical subunits, while “heterodimers” contain two nonidentical subunits. “Multimers” contain two or more subunits per functional unit and may comprise identical and nonidentical polypeptide chains.

The term “mature form” of a polypeptide or protein refers to a finally processed form of the protein, lacking precursor segments commonly referred to as signal peptides or transit peptides. Specifically, the Ncp29 polypeptide of the present invention is translated as a precursor polypeptide with an amino-terminal signal peptide of about 30 residues, involved in directing the polypeptide to the surface of the plasma membrane. The signal peptide is removed from the polypetide in the course of its cellular transport, with the final product being a surface protein that lacks the signal peptide (i.e., the “mature form” of the protein).

The term “substantially pure” refers to a preparation comprising at least 50-60% by weight the compound of interest (e.g., nucleic acid, oligonucleotide, protein, etc.). More preferably, the preparation comprises at least 75% by weight, and most preferably 90-99% by weight, the compound of interest. Purity is measured by methods appropriate for the compound of interest (e.g. chromatographic methods, agarose or polyacrylamide gel electrophoresis, HPLC analysis, and the like). Where used herein above the term “by weight” means the weight of the sample, exclusive of water and salts.

The term “substantially the same” refers to nucleic acid or amino acid sequences having sequence variation that do not materially affect the nature of the protein (i.e. the structure, stability characteristics, substrate specificity, antigenicity or immunological recognition, and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

The terms “percent identical” and “percent similar” are also used herein in comparisons among amino acid and nucleic acid sequences. When referring to amino acid sequences, “identity” or “percent identical” refers to the percent of the amino acids of the subject amino acid sequence that have been matched to identical amino acids in the compared amino acid sequence by a sequence analysis program. “Percent similar” refers to the percent of the amino acids of the subject amino acid sequence that have been matched to identical or conserved amino acids. Conserved amino acids are those which differ in structure but are similar in physical properties such that the exchange of one for another would not appreciably change the tertiary structure of the resulting protein. Conservative substitutions are defined in Taylor (1986, J. Theor. Biol. 119:205). When referring to nucleic acid molecules, “percent identical” refers to the percent of the nucleotides of the subject nucleic acid sequence that have been matched to identical nucleotides in the comparison sequence.

“Identity” and “similarity” can be readily calculated by known methods. Nucleic acid sequences and amino acid sequences can be compared using computer programs that align the similar sequences of the nucleic or amino acids thus define the differences. The Blastn and Blastp 2.0 programs provided by the National Center for Biotechnology Information (at http://www.ncbi.nlm.nih.gov/blast/; Altschul et al., 1990, J Mol Biol 215:403-410) using a gapped alignment with default parameters, may be used to determine the level of identity and similarity between nucleic acid sequences or amino acid sequences.

With respect to nucleic acid molecules, percent homology (also referred to as percent identity) may also be defined by hybridization between two single-stranded nucleic acid molecules of sufficiently complementary sequence to permit such hybridization under pre-determined stringency conditions generally used in the art. One common formula for calculating the stringency conditions required to achieve hybridization between nucleic acid molecules of a specified percent identity is set forth by (Sambrook et al., 1989):

Tₘ=81.5° C.+16.6Log[Na+]+0.41(% G+C)−0.63(% formamide)−600/#bp in duplex

As an illustration of the above formula, using [N+]=[0.368] and 50% formamide, with GC content of 42% and an average probe size of 200 bases, the Tₘ is 57° C. The Tₘ of a DNA duplex decreases by 1-1.5° C. with every 1% decrease in homology. Thus, targets with greater than about 75% sequence identity would be observed using a hybridization temperature of 42° C.

The stringency of the hybridization and wash depend primarily on the salt concentration and temperature of the solutions. In general, to maximize the rate of annealing of the probe with its target, the hybridization is usually carried out at salt and temperature conditions that are 20-25° C. below the calculated Tₘ of the of the hybrid. Wash conditions should be as stringent as possible for the degree of identity of the probe for the target. In general, wash conditions are selected to be approximately 12-20° C. below the Tₘ of the hybrid. In regards to the nucleic acids of the current invention, a moderate stringency hybridization is defined as hybridization in 6×SSC, 5×Denhardt's solution, 0.5% SDS and 100 μg/ml denatured salmon sperm DNA at 42° C., and wash in 2×SSC and 0.5% SDS at 55° C. for 15 minutes. A high stringency hybridization is defined as hybridization in 6×SSC, 5×Denhardt's solution, 0.5% SDS and 100 μg/ml denatured salmon sperm DNA at 42° C., and wash in 1×SSC and 0.5% SDS at 65° C. for 15 minutes. A very high stringency hybridization is defined as hybridization in 6×SSC, S×Denhardt's solution, 0.5% SDS and 100 μg/ml denatured salmon sperm DNA at 42° C., and wash in 0.1×SSC and 0.5% SDS at 65° C. for 15 minutes.

A “coding sequence” or “coding region” refers to a nucleic acid molecule having sequence information necessary to produce a gene product, when the sequence is expressed. A “coding sequence” may be determined indirectly from a known polypeptide sequence by understanding the genetic code. Since each amino acid is coded for by a codon containing three nucleotide bases, it is easy to ‘back-translate from a polypeptide sequence to a corresponding nucleotide sequence using a simple table of codon and their amino acid equivalents. Redundancy in the genetic code and “wobble” allow many possible “degenerate” sequences to encode the polypeptide of interest. A specific choice of a representative nucleotide sequence may be made on the basis of codon usage preference or codon bias, or degenerate sequences can be used for purposes where the ambiguity can be tolerated. Many of the commonly available molecular biology and/or molecular genetic computer packages provide a back-translation function. Other back-translation applications are available for public use or free download on the Internet.

Transcriptional and translational control sequences are DNA regulatory sequences, such as promoters, enhancers, polyadenylation signals, terminators, and the like, that provide for the expression of a coding sequence in a host cell. The terms “promoter”, “promoter region” or “promoter sequence” refer generally to transcriptional regulatory regions of a gene, which may be found at the 5′ or 3′ side of the coding region, or within the coding region, or within introns. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

The term “operably linked” or “operably inserted” means that the regulatory sequences necessary for expression of the coding sequence are arranged in a nucleic acid molecule in the appropriate positions, relative to the coding sequence, so as to enable expression of the coding sequence. This same definition is sometimes applied to the arrangement of other transcription control elements (e.g. enhancers) in an expression vector.

A “vector” is a replicon, such as plasmid, phage, cosmid or virus, to which another nucleic acid segment may be operably inserted so as to bring about the replication or expression of the segment.

The term “nucleic acid construct” or “DNA construct” refers to genetic sequence used to transform cells or organisms. The term is sometimes used to refer to a coding sequence or sequences operably-linked to appropriate regulatory sequences and inserted into a vector. This term may be used interchangeably with the term “transforming DNA”. Such a nucleic acid construct may contain a coding sequence for a gene product of interest, along with a selectable marker gene and/or a reporter gene. The transforming DNA may be prepared according to standard protocols such as those set forth in “Current Protocols in Molecular Biology”, eds. Frederick M. Ausubel et al., John Wiley & Sons, 1999. Methods of transformation are specific to the kinds of cells transformed and are well known in the art.

The term “selectable marker gene” refers to a gene encoding a product that, when expressed, confers a selectable phenotype such as antibiotic resistance on a transformed cell.

The term “reporter gene” refers to a gene that encodes a product which is readily detectable by standard methods, either directly or indirectly.

A “heterologous” region of a nucleic acid construct is an identifiable segment (or segments) of the nucleic acid molecule within a larger molecule that is not found in association with the larger molecule in nature. Thus, when the heterologous region encodes a mammalian gene, the gene will usually be flanked by DNA that does not flank the mammalian genomic DNA in the genome of the source organism. In another example, a heterologous region is a construct where the coding sequence itself is not found in nature (e.g., a cDNA where the genomic coding sequence contains introns, or synthetic sequences having codons different than the native gene). Allelic variations or naturally-occuing mutational events do not give rise to a heterologous region of DNA as defined herein. The term “DNA construct”, as defined above, is also used to refer to a heterologous region, particularly one constructed for use in transformation of a cell.

A cell has been “transformed” or “transfected” by exogenous or heterologous DNA when such DNA has been introduced inside the cell. The transforming DNA may or may not be integrated (covalently linked) into the genome of the cell. In prokaryotes, yeast, and mammalian cells for example, the transforming DNA may be maintained on an episomal element such as a plasmid. With respect to eukaryotic cells, a stably transformed cell is one in which the transforming DNA has become integrated into a chromosome so that it is inherited by daughter cells through chromosome replication. This stability is demonstrated by the ability of the eukaryotic cell to establish cell lines or clones comprised of a population of daughter cells containing the transforming DNA. A “clone” is a population of cells derived from a single cell or common ancestor by mitosis. A “cell line” is a clone of a primary cell that is capable of stable growth in vitro for many generations.

“Variant”, as the term is used herein, is a polynucleotide or polypeptide that differs from a reference polynucleotide or polypeptide respectively, but retains essential properties. A typical variant of a polynucleotide differs in nucleotide sequence from another, reference polynucleotide. Changes in the nucleotide sequence of the variant may or may not alter the amino acid sequence of a polypeptide encoded by the reference polynucleotide. Nucleotide changes may result in amino acid substitutions, additions, deletions, fusions and truncations in the polypeptide encoded by the reference sequence, as discussed below. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more substitutions, additions, deletions in any combination. A substituted or inserted amino acid residue may or may not be one represented in the genetic code. A variant of a polynucleotide or polypeptide may be naturally occurring such as an allelic variant, or a single nucleotide polymorphism (SNP) or it may be a variant that is not known to occur naturally. Non-naturally occurring variants of polynucleotides and polypeptides may be made by mutagenesis techniques or by direct synthesis.

The term “antibodies” as used herein includes polyclonal and monoclonal antibodies, recombinant antibodies, chimeric, single chain, and humanized antibodies, as well as F_{ab} fragments, including the products of an F_{ab} or other immunoglobulin expression library. With respect to the antibodies and other molecules which bind the antigens or antibodies of the invention, the terms, “specific”, “immunospecific”, “immunologically specific”, and “biologically specific” refer to antibodies or other molecules which bind to one or more epitopes of a protein or of interest, but which do not substantially recognize and bind other molecules in a sample containing a mixed population of antigenic biological molecules. “Secondary” antibodies are antibodies which are specific for other antibodies, and include antibodies specific for the antibodies of a particular class, such as anti-IgG antibodies. Anti-species specific antibodies may also be considered “secondary” antibodies, for example, mouse anti-bovine antibodies are antibodies derived from the immune system of the mouse, and which have specificity for antigens which are antibodies from cows, but which do not specifically bind to antigens which are antibodies from other species. “Secondary binding components” include secondary antibodies, and further include other such molecules as may specificity of affinity for the Ab-Ag complexes, or for the antibodies of the invention. Examples of secondary binding components include secondary antibodies, detectable marker-linked secondary antibodies, Protein G, Protein A and the like.

Description

The emergence of neosporosis as a disease of economic and agronomic importance has created the need for a highly sensitive method of diagnosing this disease and of detecting the presence of the causative pathogen, Neospora caninum. The present invention provides an improved method for detection of antibodies to N. caninum and diagnosis of the disease condition, neosporosis, based on a novel recombinant antigen. The description set forth below first describes the recombinant antigen of the invention, as well as polynucleotides encoding the antigen and antibodies immunologically specific for the antigen. The improved immunological assay that utilizes the recombinant antigen is then described.

In accordance with the present invention, an isolated nucleic acid encoding a polypeptide substantially the same as the mature portion of the protein Ncp29 is provided. The protein, Ncp29 (also known as Nc-p36, or p36) is produced as a precursor whose sequence is available as GenBank Accession Number AF132217 (cDNA is SEQ ID NO:2; encoded protein is SEQ ID NO:4) In one embodiment, the nucleic acid is provided as a cDNA. In another embodiment, the coding sequence of the nucleic acid has been to modified so that the expressed polypeptide contains a polyHis tag at the C-terminus to facilitate purification. Other embodiments contain additional or alternate sequence modifications, known to those skilled in the art, which provide convenient ‘handles’ for purification of the expressed protein. In another embodiment, the nucleic acid contains one or more restriction endonuclease cleavage sites engineered at strategic locations. Strategically engineering such sites is routine to those skilled in the art. In one embodiment, the restriction endonuclease cleavage sites allow the nucleic acid to be inserted into a vector of choice. In a preferred embodiment, the vector of choice is an expression vector. In another preferred embodiment, the restriction endonuclease cleavage sites are placed such that the coding sequence of the nucleic acid when inserted into an appropriate expression vector is in the proper reading frame (or ‘in frame’) with respect to the expression requirements of the vector, such that expression of the coding sequence is enabled. In another preferred embodiment, the restriction endonuclease cleavage sites are NdeI and XhoI. In yet another preferred embodiment, the nucleic acid has a sequence which is substantially the same as that given in SEQ ID No:1, wherein the 5′ hexanucleotide comprises an Nde 1 site, the first three bases of which are not expressed in the protein, and further wherein the 3′ end comprises an Xho1 site (encoding amino acid residues LE) that links the protein coding sequence to a poly-histidine coding sequence). In an exemplary embodiment, the nucleic acid has a sequence which is that of SEQ ID No:1 or that portion of SEQ ID NO:2 that encodes the mature portion of Ncp29.

Also provided in accordance with the present invention is an isolated antigen substantially the same as that of the mature form of the Neospora caninum protein known alternately as Ncp29, Nc-p36 or p36 (the mature portion of SEQ ID NO:4, residues 31-315). This antigen in its recombinant form is referred to herein as rNcp29. The recombinant antigen, in a preferred embodiment retains all of the antigenic properties of its naturally occurring counterpart. In another preferred embodiment, the recombinant antigen has greater binding specificity, affinity, and/or avidity than does its naturally occurring counterpart, so as to provide increased sensitivity for applications in detecting antibodies to Neospora.

In various embodiments, the antigen may be altered or modified by methods known to those skilled in the art, provided that the antigenicity is maintained. In one embodiment, modifications include conservative amino acid substitutions, site-directed mutagenesis-mediated alterations in specific amino acids, or specific additions or deletions of amino acids not critical to maintain antigenicity. In one embodiment, the antigen is modified by methods known in the art to increase antigenicity. In a highly preferred embodiment, the antigen is consistently, and with high specificity and avidity, recognized by anti-Neospora antibodies in the serum of naturally infected animals. In another embodiment, the amino acid sequence of the antigen is substantially the same as that of SEQ ID NO:3 or residues 31-315 of SEQ ID NO:4. In a preferred embodiment, the amino acid sequence of the antigen is that given in SEQ ID NO:3 or residues 31-315 of SEQ ID NO:4.

Also provided in accordance with the present invention is a recombinant expression system comprising the isolated nucleic acid of the invention. In a preferred embodiment, the expression system allows large-scale production of the antigen. In another preferred embodiment, the expression system is a bacterial, yeast or insect expression system as are known to those skilled in the art. Such expression systems are widely used and the criteria for selecting them are known to those skilled in the art. In a preferred embodiment, the expression system is inducible. In one embodiment, the expression system produces the antigen in granules or aggregates, in another embodiment the antigen is secreted into the medium. In a preferred embodiment, the expression system is a bacterial system. In a highly preferred embodiment, the expression system comprises the pET vectors (Novagen).

The present invention also provides the protein product of such an expression system—the recombinant antigen, rNcp29. The recombinant antigen can be purified by a variety of means known to those skilled in the art of protein purification. In a preferred embodiment, the recombinant antigen is produced in granules or secreted into the medium to facilitate purification. In one embodiment the recombinant antigen contains within its amino acid sequence a combination of amino acids which provide a ‘handle’ to further facilitate purification. Examples of such ‘handles’ are known to those in the art. In one embodiment, the handle provides a means by which the recombinant antigen may be affinity purified. In one embodiment, the ‘handle’ is an antigenic epitope and the affinity purification is via an antibody of the invention. In a preferred embodiment, the handle is provided by means of a polyhistidine (polyHis) tag at the C-terminus of the polypeptide and the affinity purification is via immobilized metal affinity chromatography.

In various embodiments, the recombinant antigen may be altered or modified by methods known to those skilled in the art, provided that the antigenicity is maintained. In one embodiment, modifications to the recombinant antigen include conservative amino acid substitutions, site-directed mutagenesis-mediated alterations in specific amino acids, or specific additions or deletions of amino acids not critical to maintain immunogenicity and antigenicity. In one embodiment, the recombinant antigen is modified by methods known in the art to increase antigenicity. In a highly preferred embodiment, the recombinant antigen is consistently, and with high specificity and avidity, recognized by antibodies in the serum of naturally infected animals. In another embodiment, the recombinant antigen is highly immunogenic and generates a substantial immune response and a high antibody titer in exposed animals. In another embodiment, the amino acid sequence of the recombinant antigen is substantially the same as that of SEQ ID NO:3 or residues 31-315 of SEQ ID NO:4. In another embodiment, the nucleic acid encoding the amino acid sequence of the recombinant antigen is modified by altering the codon usage in accordance with a codon usage preference table (or software program encompassing the same data) such that the expression is optimized for the organism in which it is being expressed. The optimization of codon usage to enhance protein expression is known to those skilled in the art.

Isolated antibodies to the antigen are also provided in accordance with the present invention. The antibodies may be derived from and contained in isolated biological fluids. Further, they can be partially or extensively purified from a crude source of such antibodies. In one embodiment, antibodies of the invention are polyclonal antibodies, in another they are monoclonal. In a preferred embodiment the antibodies of the invention recognize at least one epitope which is unique to the antigen or recombinant antigen of the invention. In a preferred embodiment, the epitope recognized by the antibody is highly immunogenic and creates a immune response which generates a high titer of antibodies in an animal injected with the antigen. In one embodiment of the invention, the antibodies of the invention can be purified by use of affinity purification using the recombinant antigen. In another embodiment, the antibodies can be used as affinity ligands to purify the recombinant antigen. It is contemplated that the antibodies to the antigen of the invention are used in immunological assays, detection systems, affinity methods, diagnostic kits, rapid detection methods, and other such applications as are known to those in the art. Immunologically active fragments, portions, subunits or polypeptide chains of the antibodies, and partially purified or purified antibodies may also be used in the same manner. The antibodies of the invention may be of any class and further may comprise multimers of such molecules. It is further contemplated that the antibodies of the invention may also be cloned as to their binding fragments and produced recombinantly. In addition, the antibodies may be covalently or noncovalently attached to fluorescent tags, enzymes, radiolabel, biotin, or other ligands, or other molecules which directly or indirectly provide a means by which said antibodies can be readily detected. Such antibodies are useful in performing direct immunological assays for the detection of the antigens of the invention The uses described herein with respect to the antibodies of this invention are intended to illustrate not to limit their application. Other uses and adaptations of the antibodies of the present invention will be obvious to the those skilled in the art and are considered part of the present invention.

The main feature of the present invention is an immunological assay method for the detection of anti-Neospora antibodies in biological fluids. The assay method requires the recombinant antigen of the invention. Any immunological assay utilizing this recombinant antigen is contemplated for use in the present invention. For instance, in one embodiment, the assay is an indirect immunofluorescence test. In another embodiment, the assay is a dot blot, slot-blot or other blotting or membrane-transfer application. Another embodiment is conducted as a Western blot or automated Western blot. Other assays types contemplated for use with the present invention include those conducted as immunodiffusion, immunoelectrophoretic methods, immunoprecipitation, and other indicator-labeled immunoassays, including for example: enzyme immunoassay (EIA); fluorescence immunoassay (FIA); radioimmunoassay (RIA); enzyme multiplied immunoassay technique (EMIT); and fluorescence polarization immunoassay (FPIA). In certain embodiments, the antigen is labeled with a detectable marker, while in other embodiments, an indirect method is used for detecting an Ab-Ag complex formed during the assay. Such indirect methods include use of a detectable marker-labeled secondary antibody.

In a preferred embodiment, the assay is conducted as an enzyme linked immunosorbent assay (ELISA). In a preferred embodiment the assay is conducted within a well of an ELISA plate, preferably a high binding capacity ELISA plate, with the recombinant antigen bound to the well of the plate. The binding of antigens to wells of ELISA plates and other surfaces is well-understood by those of skill in the art. To minimize nonspecific binding of further proteins to the wells, a blocking agent is used, which comprises a commonly available protein customarily used to block nonspecific protein. The selected blocking agent should be one that is unlikely to contain antigenic epitopes which would interfere with the detection of antibodies specific for the antigen of the invention. In a preferred embodiment, bovine serum albumin (BSA) is the blocking agent.

After a proper blocking step, such as incubation with BSA at 37° C. for 1 hour or overnight at 4° C., the bound antigen is contacted, under appropriate conditions, by antibody specific for the antigens of the invention, or by isolated biological fluids which may contain such antibodies. Such biological fluids are those normally considered to contain immunoglobulins or immune cells, particularly blood and fractions or portions thereof. In one embodiment, isolated serum or blood is tested. In other embodiments, other biological fluids, commonly known to contain immunoglobulins or immune cells are tested. Included among these fluids are blood fractions, lymph, pus, colostrum, placental fluids, mucous discharges and milk. Other fluids such as urine, saliva, sweat, lachrymous fluids, gastric fluids, stomach contents, digestive fluids, intestinal fluids, synovial fluids, ascitic fluids, peritoneal fluids, pericardial fluids, pleural fluids, cerebrospinal fluids, labyrinthine fluids, reproductive fluids and the like could potentially contain antibodies to the antigens of the invention under specific circumstances and are thus contemplated as biological fluids to be tested with the assays of the present invention. Such fluids may be from any biological source whatever, including healthy, suspected diseased, frankly diseased and deceased animals. The biological fluids to be tested may also be derived from animals which are intentionally infected with Neospora caninum, or which are injected with the antigen or recombinant antigen of the invention. Many accepted methods for collection of such fluids are known to those skilled in the art. In one embodiment the biological fluids for testing in the assays of the present invention derive from certain animals; examples of such animals of interest include cattle (dairy or beef), dogs, sheep, goats, horses, deer and humans, as well as any other farm animals, meat animals, dairy animals or zoo animals that may require such testing. Biological fluids from these and other animals may be considered as sources of antibodies of the present invention, in addition to being considered as sources for testing by the assays of the invention. The isolated biological fluids can be used to detect the presence of antibodies to Neospora or for source material from which to diagnose neosporosis.

The conditions for antigen binding, blocking agent addition and antibody addition, and the like, typically comprise a buffer such as phosphate-buffered saline (PBS). The buffer may contain a surfactant, for example the nonionic surfactant, Tween 80. In one embodiment, the conditions for contact include incubation at a temperature from room temperature up to about 37° C., for a time adequate to allow Ab-Ag complex formation, under conditions of pH and ion concentrations that permit or promote formation of Ab-Ag complexes. In other embodiments conditions are lower temperatures and longer times, for example 4° C. overnight. Following this incubation, Ab-Ag complexes are separated from the unbound antibodies or from remaining portions of a biological fluid being tested. In a preferred embodiment, the complexes bound to a solid support such as an ELISA well plate, are rinsed free of such unbound material. leaving only bound antigen and bound Ab-Ag complexes. In this embodiment, the rinse solution preferably is the same buffer used for the binding the antigen to the wells and for the addition of the blocking agent. In another embodiment, the complexes may be present in solution or may be bound to a separable support, such as a microbead, and thus separated from the remaining biological materials by conventional means, e.g., centrifugation or magnetic separation.

The Ab-Ag complexes are detected by any of the available methods known to those skilled in the art. In one embodiment, the Ab-Ag complexes are contacted by a secondary binding component that has biological binding specificity for Ab-Ag complexes. The secondary binding components of the present invention include antibodies (referred to herein as “secondary antibodies”) made in one species to antibodies from another species, which bind to the antigens of the invention, or to the Ab-Ag complexes formed in the assay. Alternatively, the secondary binding component can be an antibody-binding molecule such as Protein A or Protein G. In a preferred embodiment, Protein G is used as the secondary binding component. In another preferred embodiment, a secondary antibody, specific for antibodies from the species of animal from which the biological fluid being tested is derived, is used. The Ab-Ag complexes are contacted with the secondary binding component under conditions suitable for binding the secondary binding component with the Ab-Ag complex. Conditions for this binding are similar to those provided above for Ab-Ag complex formation.

In addition to its biological specificity for antibodies, the secondary binding component comprises a detectable marker such as an enzyme, a ligand, a fluorescent tag, a luminescent tag, a radiolabel, gold-labeled, or the like, to enable detection of the secondary binding component. These secondary binding components and detectable marker-linked secondary binding components are well known to those skilled in the art. In one preferred embodiment, the secondary binding component contains an enzyme covalently attached such that the enzyme activity is retained. Upon incubation with a specific substrate, the enzyme catalyzes the formation of product, and the loss of substrate and/or the accumulation of product is measured. In a preferred embodiment, the enzyme is horseradish peroxidase (HRP), alkaline phosphatase, such as from calf intestine, or β-galactosidase, such as that from E. coli. In another preferred embodiment, the substrate is chosen for its quantitative yield of a colored, fluorescent or luminescent reaction or other product which is readily detected.

In the assays described herein, the use of standard curves and/or controls allows the determination in quantitative or qualitative terms of the amount of antibody present in a biological fluid being tested. In cases where the amount of antibody present exceeds the discrimination ability of the method, serial dilutions of the biological fluid being tested are prepared and subsequently tested by the immunological assay method of the invention. In a preferred embodiment, a titer is calculated from the data from serial dilutions of a sample. The calculations and determination of titer is commonly known to those skilled in the art. Using the recombinant antigen of the invention, the immunological assay method of the invention provides greater sensitivity, and/or higher signal-to-background noise ratio than do previously available immunological assay methods for the detection of Neospora caninum.

The exemplary method set forth below has demonstrated very good sensitivity for the detection of anti-Neospora antibodies in biological fluids, as described in greater detail in the Examples. The assay is conducted as an indirect ELISA, using a high binding capacity ELISA plate. An aliquot of a solution of purified rNcp29 (e.g., 100 μl at 10 μg/ml in PBS is added to each well of the plate. Following incubation for, e.g., 1 h at 37° C. (or a longer time at a lower temperature), the plate is rinsed appropriately (e.g., 3 times) with PBS plus 0.05% Tween 20. The wells are then blocked for 1 h at room temperature (or for appropriately longer or shorter times at lower or higher temperatures, respectively) with 1% γ-globulin-free BSA. The test sample comprises serum from animals to be tested, which is diluted 1:500 in PBS plus 0.05% Tween 20 and 0.1% BSA. Aliquots (e.g., 100 μl) are added to wells of the ELISA plate and incubated 2 h at room temperature. After binding, the wells are rinsed (e.g., 3 times) with PBS plus 0.05% Tween 20. Protein G-horseradish peroxidase (HRP) conjugate diluted according to suppliers' instructions (e.g., 1:2500) in PBS plus 0.05% Tween 20 and 0.1% BSA is added (e.g., 100 μl) to each well and incubated 2 h at room temperature or for appropriately shorter or longer periods depending on temperature. The plate is then rinsed well (e.g., 4 times) in PBS plus 0.05% Tween 20 and 0.1% BSA. The chromogenic substrate, o-phenylenediamine (OPD) dihydrochloride (Sigma), dissolved in 0.05 M phosphate-citrate buffer to a concentration of 0.4 mg/ml, is added (200 μl) to each well. After a timed incubation (e.g., 10 min), the reaction is stopped, and the absorbance is measured at 490 nm in an ELISA plate reader. Samples tested in duplicate may be averaged and reported as mean values. The foregoing exemplary assay method detected positive titers (optical density (O.D.)>1.2 at 1:500 or greater dilution) in 13 of 13 infected cattle while 13 of 13 control animals were negative (O.D.<0.5 at 1:500). Additionally, sera from animals that were infected with T. gondii or S. cruzi did not cross-react to recombinant Ncp29 in the ELISA assay.

Also provided in accordance with the present invention is a method of assessing the disease status of an animal, or group of animals with respect to the Neospora caninum infection, neosporosis. In one embodiment of this method, a titer of circulating rNcp29-specific antibodies is determined by an above method for each animal. The titer is then compared to titers of known positive and known negative test samples determined in an analogous fashion by the immunological assay of the invention. Further correlation may be done with the titers previously determined from other positive or negative animals, from paired comparison animals, from other animals in the same group, or from data from assay standard curves. In a preferred embodiment, criteria are established, based on the foregoing information, for determining an animals status with respect to the disease, neosporosis. In another preferred embodiment, the criteria established allow each animal to be classified as for example, ‘healthy’, ‘infected’ or ‘suspect’, based on titer as determined by the above methods. Such an assessment is useful in allowing preventive veterinary measures, disease control, proper epidemic containment, animal isolation, animal culling, animal breeding and other animal management decisions to be made in an informed manner. In another embodiment, pooled samples of biological fluids from herds or groups of animals may be tested as a rapid screen of the disease status of the group. Criteria may be established for determining when subsampling or individual animal testing is required based on the results of pooled biological fluids. Such a pooled sample would be a useful screen, for example, where prior individual testings have proven negative, or where other evidence suggests absence of infection in the group. In another embodiment of the invention, routine analysis of each animal's health status with respect to neosporosis is checked periodically in conjunction with other routine monitoring or health assessments. In a preferred embodiment, results of such monitoring are a part of the animal's permanent health, management and/or regulatory records.

In another aspect of the invention, kits for the immunological detection of Neospora caninum and/or for the diagnosis of neosporosis are provided. Diagnostic kits and assay kits are widely known in the art. They are useful to practitioners of the art, as well as those in ancillary arts, who require or desire additional ease of use. In a preferred embodiment, the kits of the present invention include one or more of the following: control antiserum from a known infected animal, or pooled from known infected animals; control antiserum from an uninfected animal, or pooled from uninfected animals; the recombinant antigen of the invention; a blocking agent; buffers, including surfactants; a secondary binding component as described above; a detection system for detecting the antibody-antigen complexes; controls for colorimetric, fluorometric or luminous detection systems; instructions for use; and certificates of quality control for various components.

In summary, a sensitive and specific assay for detecting antibodies to N. caninum in the serum of infected animals, has been developed in accordance with the present invention. The method is based on screening serum from animals for the presence of antibodies specific to recombinant Ncp29. The recombinant Ncp29-based immunological assay provides a specific and sensitive means for detecting neosporosis in cattle and is useful for detecting N. caninum infection in other susceptible animals such as dogs and horses.

Although bovine testing is exemplified herein, it is clear that such a method will be applicable to other animals. For example, the Ncp29 ELISA assay is contemplated to also be useful for diagnosis of neosporosis in dogs. Canids are the definitive host for N. caninum, and detection of infections in dogs is of practical value in two ways. First, adequate control of neosporosis on cattle farms will ultimately depend on disrupting transmission. Good management practices to prevent infection are aided by determining which animals are infected, allowing the option of culling or treating animals as deemed necessary. Secondly, neosporosis is a significant cause of neurological disease in dogs. Early detection of these infections, combined with adequate treatment are key to preventing recurrent episodes of congenital infection and paralysis that have been known to occur in dogs.

Similarly, the assay can easily be adapted to examine the prevalence of infection in other animals of economic importance including horses, goats, sheep and other livestock animals. While the present assay relies on recognition of the serum antibodies with protein G, which does not react equally well to immunoglobulins in all species, modification to include a species-specific detection is understood to be easily incorporated to allow detection of a range of host species. The detection of species-specific antibodies to N. caninum could also be conducted by a variety of formats not limited to the specific ELISA described here but also employing Western Blotting, IFA, and immunohistochemistry.

Recently it has been reported that the gene encoding the p29 antigen is entirely conserved among a variety of isolates of N. caninum isolated from bovine and canine hosts from North America and Europe (GenBank accession numbers AF141960, AF141961, AF141962, AF141963). Such a high degree of conservation indicates that the rNcp29 immunological assay will be widely applicable to a range of different isolates that could potentially infect different hosts species. N. caninum is generally not considered an agent of human infection, yet exposure to humans by consumption of animal tissues and close contact with infected animals may pose a risk of infection. Indeed, a recent survey indicated positive serological titers in several humans suggesting that additional testing is warranted. The interest in emerging pathogens, particularly those which may be food-borne, supports such screening, and it is likely that the rNcp29-based immunological assays provided in accordance with the present invention will provide a sensitive assay to conduct such serological surveys.

Additional recent evidence further indicates that the Ncp29 antigen is conserved among other members of the genus Neospora. Specifically, characterization of Neospora hughesi, a species of Neospora that infects horses, indicates conservation of the Ncp29 antigen. Consequently, the Ncp29 antigen and immunological assays and kits of the present invention will be of use for the detection of Neospora species other than N. caninum that infect animals and that may infect humans.

The following examples are provided to describe the invention in greater detail. They are intended to illustrate, not to limit, the invention.

### EXAMPLE 1

### ELISA for the Detection of Neospora Infections in Bovine Sera

Materials and Methods

Recombinant protein production. The open reading frame of the gene encoding Ncp29 (GenBank accession AF132217) was amplified with Klentaq-LA polymerase (Clontech) from genomic DNA of the Nc-1 strain of N. caninum . The sequence of the forward primer used was:

5′GATCCATATGTCAGAAAAATCACCTCTAC3′ (SEQ ID NO:5). The sequence of the reverse primer used was: 5′GATCCTCGAGCGCTATCGAGCCTACG3′ (SEQ ID NO:6). The PCR-amplified fragment was ligated into NdeI/XhoI-digested pET-22b expression vector (Novagen) using NdeI and XhoI restriction sites that were incorporated into the amplification primers. The resulting recombinant antigen expression plasmid, prNcp29, was used to transform BL21 E. coli. A clone that expressed substantial quantities of recombinant Ncp29 was selected for further use. Following induction with IPTG, and expression of the protein, recombinant Ncp29 (rNcp29) was extracted from inclusion bodies using 6M urea, and the protein was purified by nickel-affinity chromatography according to the manufacturer's protocol (Novagen). The purified protein was concentrated using Centricon-10 columns (Amicon) and the protein concentration was determined by a calorimetric protein assay (Pierce) prior to storage at −80° C. until use. The expressed recombinant protein, rNcp29, lacks the predicted 30-amino acid signal peptide at the N-terminus and the last four amino acids of the carboxyl terminus of the native protein and contains a C-terminal histidine tag.

SDS-PAGE and Western blotting. Tachyzoites of the Nc-1 strain of N. caninum were propagated in human fibroblasts and purified as described previously (Howe, et al., (1998) Infect. Imun. 66; 5322-5328). Parasite cell pellets or lysates of E. coli expressing recombinant Ncp29 were resuspended in SDS PAGE sample buffer and separated on 10% acrylamide gels. Proteins were transferred to nitrocellulose by semi-dry electrophoresis in Tris-glycine pH 8.3 and Western-blotted using horseradish peroxidase (HRP)-conjugated secondary antibodies (Jackson ImmunoResearch Labs) and chemiluminescence (Pierce) as described (Howe, et al., 1998, supra). Primary antisera consisted of the previously described mAb 6C11 which reacts to Ncp29 and sera from mice that were chronically infected with N. caninum.

ELISA. Purified rNcp29 was diluted to 10 μg/ml in phosphate-buffered saline (PBS) and 100 μl was added per well in high-binding-capacity ELISA plate (Corning), and incubated for 1 hr at 37° C. or overnight at 4° C. The plate was rinsed three times with PBS containing 0.05% Tween 20 and blocked for 1 hr with PBS containing 0.05% Tween 20 and 1% γ-globulin-free bovine serum albumin (BSA) (Sigma, St. Louis, Mo.). Antisera were diluted to 1:500 in PBS containing 0.05% Tween 20 and 0.1% BSA, and 100 μl was added to duplicate wells of the ELISA plate, and incubated for 2 hr at room temperature (RT). The wells were rinsed three times with PBS containing 0.05% Tween and incubated for 2 hrs at RT with 100 μl of Protein G-HRP conjugate (Pierce) diluted to 1:2500 in PBS containing 0.05% Tween 20 and 0.1% BSA. Finally the plate was rinsed four times with PBS containing 0.05% Tween 20. The chromogenic substrate, o-phenylenediamine (OPD) dihydrochloride (Sigma), was dissolved in 0.05 M phosphate-citrate buffer to a concentration of 0.4 mg/ml, and 200 μl of substrate was added into each well. After a 10 min incubation, the reaction was stopped with 50 μl of 2 M H₂SO₄, and the A₄₉₀ was measured in an ELISA plate reader. Samples were performed in duplicate and are reported as mean values.

Bovine sera. Pre- and post-infection sera were obtained from three cows that were experimentally infected with the BPA-1 isolate of N. caninum. Serum samples were also obtained from ten each known positive and negative animals; positive or negative sero-status was determined by immunofluorescence assay (IFAT). A complete description of the sera and the clinical histories for the animals can be found in Table 1. To assess the specificity of the rNcp29 ELISA, serum samples were also obtained from bovine animals experimentally infected with either Sarcocystis cruzi or T. gondii.

TABLE 1
Reciprocal titers of cattle serum samples from animals with confirmed
positive versus negative infections with N. caninum. Titers were
determined by IFAT and by Ncp29 ELISA.

| Sample # | Animal # | Diagnosis | IFAT | ELISA |
|---|---|---|---|---|
| N1 | 25Z | Negative^{a} | <80 | <100 |
| N2 | 66/74 | Negative | <80 | <100 |
| N3 | 369 | Negative | <80 | <100 |
| N4 | 370 | Negative | <80 | <100 |
| N5 | 405 | Negative | <80 | <100 |
| N6 | 2290 | Negative | <80 | <100 |
| N7 | 354 | Negative | <80 | <100 |
| N8 | 374 | Negative | <80 | <100 |
| N9 | 398 | Negative | <80 | <100 |
| N10 | 364 | Negative | <80 | <100 |
| P1 | | Positive^{b}/Calf^{c} | 20480 | 16,000 |
| P2 | | Positive/Calf | 20480 | 32,000 |
| P3 | 1088 | Positive | 20480 | 32,000 |
| P4 | 2744 | Positive | 1280 | 16,000 |
| P5 | 48291 | Positive/Calf | 1280 | 16,000 |
| P6 | 5629 | Positive | 10240 | 32,000 |
| P7 | | Positive | 640 | 8,000 |
| P8 | 522 | Positive | 160 | 8,000 |
| P9 | 927 | Positive | 640 | 8,000 |
| P10 | 3923 | Positive | 640 | 8,000 |
| Pre1 | 412 | Pre-inoculation | <80 | ND |
| Post1 | 412 | Post-inoculation | 10240 | ND |
| Pre2 | 7 | Pre-inoculation | <80 | ND |
| Post2 | 7 | Post-inoculation | 10240 | ND |
| Pre3 | A114 | Pre-inoculation | <80 | ND |
| Post3 | A114 | Post-inoculation | 10240 | ND |

^{a}Negative refers to negative immunoperoxidase or absence of detectable parasites in tissues by histological examination.
^{b}Positive indicates positive immunoperoxidase and detectable parasite clusters, free tachyzoites or cysts were detected in the tissues.
^{c}Calves were infected congenitally and were sampled post-colostrum.
^{d}(5)
^{e}reciprocal titers
ND, not done

### RESULTS AND DISCUSSION

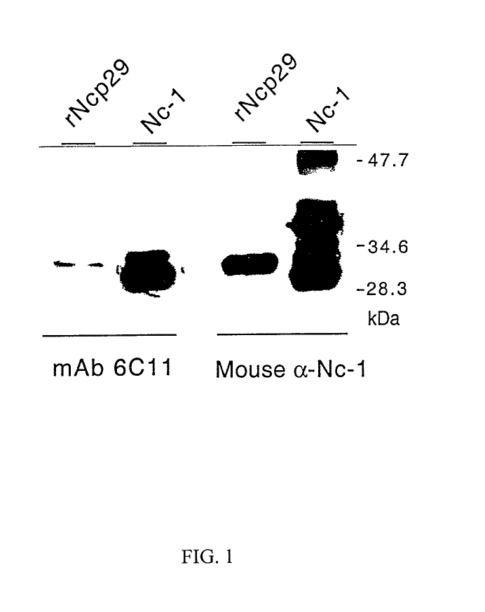

To assess the immunoreactivity of rNcp29, the recombinant protein was expressed in E. coli as a His-tagged fusion protein and purified by nickel affinity chromatography. Recombinant Ncp29 was readily recognized in Western blots by antisera from Neospora-infected mice and by the murine monoclonal antibody 6C11, which had been previously selected for its reactivity to native Ncp29 (Howe et al., 1998, supra (FIG. 1). The preservation of antigenicity of rNcp29 contrasts with previous findings that the homologous protein SAG1 in T. gondii is misfolded when expressed in E. coli such that the antigen is not readily recognized by antibodies to the native protein (Kim, et al. (1994) Infect. Imun. 62: 203-209). Conformationally-correct SAG1 from T. gondii has been produced in mammalian cells and in yeast, although these expression systems can lead to altered secondary modifications such as glycosylation (Kim et al., 1994, supra). The observation that rNcp29 produced in E. coli is recognized by antisera to the native protein suggests it is folded adequately to retain its antigenic nature. This is observation is consistent with the findings of Sonda et al. (1998, supra) who found that a recombinant fragment of the immunodominant surface antigen, p36, from Neospora caninum was expressed in E. coli and subsequently recognized by anti-p36 antibodies upon Western blotting. Consequently, we decided to evaluate the potential use of rNcp29 for specific detection of neosporosis by testing for antibodies to the protein in sera of Neospora-infected versus non-infected animals.

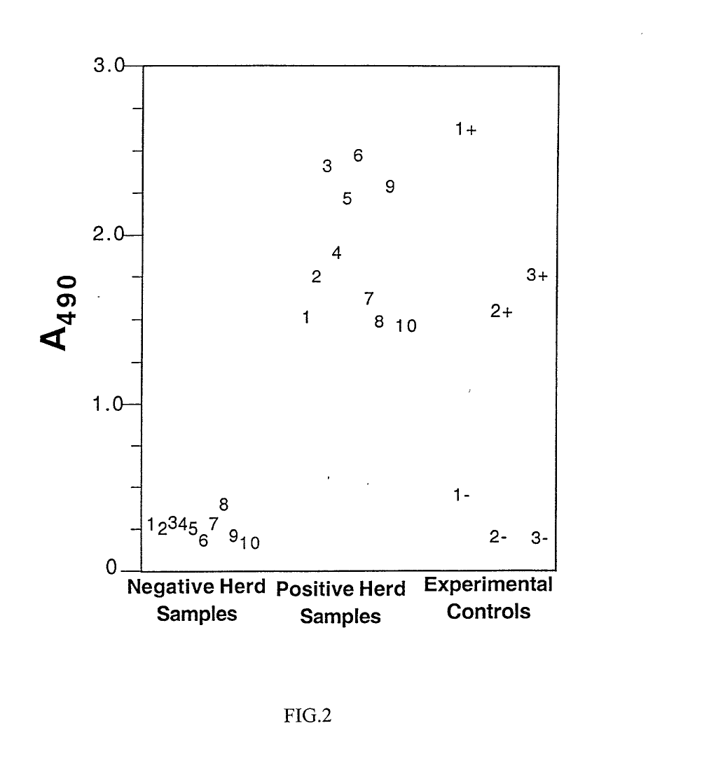

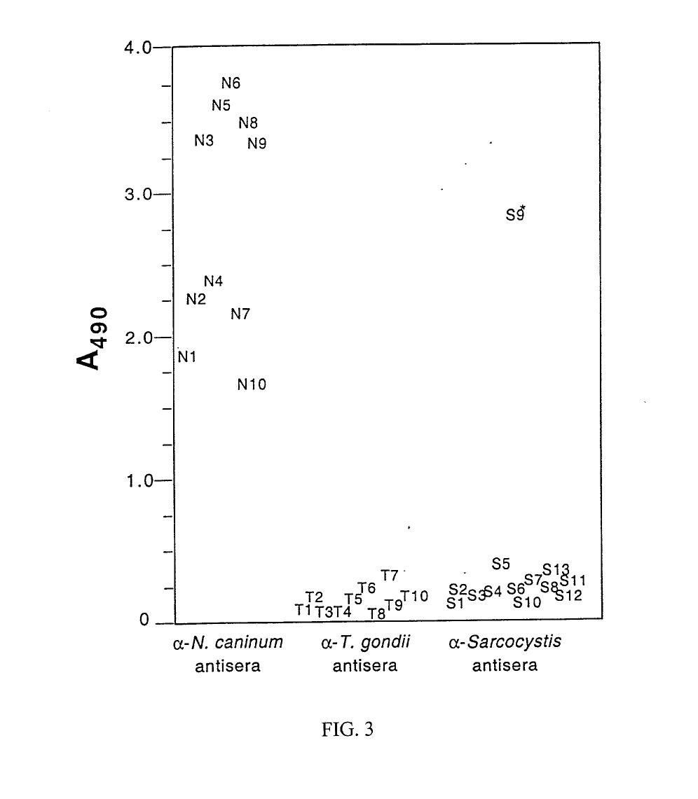

A collection of 26 bovine sera which had been previously characterized by indirect fluorescent antibody test (IFAT) for anti-N. caninum antibodies using rNcp29 (Table 1) were tested by the ELISA method. These samples were obtained from pair-wise pre- and post-infection sera collected from three animals that were experimentally infected by inoculation with the BPA-1 isolate of N. caninum. Additional samples consisted of ten confirmed cases of neosporosis and ten healthy, serologically negative cows. Several of the known positive animals had previously aborted N. caninum positive fetuses (P3, P4, P7, P8, P9, P10) and several were samples from calves that were confirmed, by histological examination, to be infected (P1, P2, and P5) (Table 1). The rNcp29 ELISA clearly distinguished the antisera of animals that had been experimentally or naturally infected with N. caninum from antisera of the non-infected animals (FIG. 2). The absorbance (A₄₉₀) for the positive samples ranged from O.D. 1.5 to 2.7 at a dilution of 1:500, while the negative samples were all below O.D. 0.5. Serial dilution of serum samples was used to determine the end-point titers, as defined by the reciprocal of the dilution where the positive value was still two-fold above the negative value of the ten pooled negative animals (Table 1). Although in most tests the titers obtained by the rNcp29 ELISA and IFAT were similar, the rNcp29 assay was for example, considerably more sensitive, detecting elevated titers, relative to the IFAT, in animals P7, P8, P9 and P10. Despite this increased sensitivity, there was no significant increase in ELISA titers of the negative animals relative to the IFAT. Most previous serological assays have been significantly hampered by the cross-reaction of Neospora antigens with closely related parasites such as T. gondii, or have lacked sensitivity. Therefore, an additional 23 bovine antisera from animals that had been experimentally infected with either T. gondii or Sarcocystis spp. were also tested with the rNcp29 ELISA. With the exception of a single animal that had been experimentally infected with Sarcocystis (sample S9), all of the control antisera exhibited an A₄₉₀ O.D.<0.5; by comparison, the known positive animals listed in Table 1 exhibited values of O.D.>1.5 (FIG. 3). Further examination of the sample from animal S9 revealed that this animal had an elevated anti-Neospora titer using a variety of assays, thus indicating that it had previously been naturally infected with N. spp.

Despite the similarity with the related gene product SAG1 that occurs in T. gondii, no antigenic cross-reaction between N. caninum and T. gondii l using Ncp29 was detected. Animals infected with N. spp. reacted strongly to Ncp29 while those infected with T. gondii showed negative ELISA titers. Ncp29 and SAG1 (also known as p30) from T. gondii share approximately 50% identity at the amino acid level, and both contain 12 cysteine residues that are predicted to be involved in conformational folding of the protein, a property that is likely important for the highly antigenic nature of SAG1. Following infection with T. gondii, antibodies to SAG1 appear very early and include IgM, IgA as well as IgG classes, making this antigen ideally suited for detection of acute infection. Due to the unique antigenicity, surface location, and abundant expression in N. caninum, Ncp29 provides an excellent antigen for detection of early infection during outbreaks of neosporosis.

The present invention is not limited to the embodiments described and exemplified above, but is capable of variation and modification within the scope of the appended claims.

We claim:

1. An isolated nucleic acid molecule encoding an antigenic polypeptide comprising a mature form of an Ncp29 protein from Neospora caninum.

2. The isolated nucleic acid molecule of claim 1, encoding a polypeptide selected from the group consisting of:
a polypeptide having residues 31-315 of SEQ ID NO:4; and
a polypeptide having a sequence greater than 80% identical to SEQ ID SEQ ID NO:4 and having equivalent antigenicity as the polypeptide having SEQ ID NO:4.

3. The isolated nucleic acid molecule of claim 2, selected from the group consisting of:
a portion of SEQ ID NO:2 that encodes SEQ ID NO:4;
a nucleic acid molecule greater than 80% identical to the portion of SEQ ID NO:2 that encodes SEQ ID NO:4, which further encodes a polypeptide having equivalent antigenicity as the polypeptide having SEQ ID NO:4; and
a nucleic acid molecule that hybridizes under moderately stringent hybridization conditions with the portion of SEQ ID NO:2 that encodes SEQ ID NO:4, which further encodes a polypeptide having equivalent antigenicity as the polypeptide having SEQ ID NO:4.

4. The isolated nucleic acid molecule of claim 1 wherein the encoded polypeptide lacks 4 carboxy-terminal amino acids of the mature form of the Ncp29 protein, and further comprises a polyHis tag.

5. The isolated nucleic acid molecule of claim 4, which encodes a polypeptide having SEQ ID NO:3.

6. The isolated nucleic acid molecule of claim 5, which comprises SEQ ID NO:1.

7. A recombinant protein expression system comprising the isolated nucleic acid molecule of claim 1.

8. A recombinantly-expressed antigenic protein produced by the recombinant expression system of claim 7.

9. The recombinantly-expressed antigenic protein of claim 5 expressed in an organism selected from the group consisting of bacteria, yeast, fungi, insects, and mammals.

10. An isolated antibody (Ab) comprising a binding site which specifically recognizes one or more epitopes of the protein of claim 8.

11. An immunological detection system for the detection of Neospora infections comprising the antigenic protein of claim 8.

12. An immunological assay method for the detection, in a biological fluid, of antibodies specific for Neospora spp. comprising the steps of:
a) providing the recombinant antigen of claim 8;
b) contacting the antigen with a biological fluid to be tested, under conditions wherein antibodies present in the biological fluid, with specificity for the recombinant antigen, bind to the antigen to form antibody-antigen complexes;
c) separating unbound antibody, and other components of the biological fluid from the antibody-antigen complexes;
d) detecting the antibody-antigen complexes; and,
e) measuring the amount of detected antibody-antigen complex, the amount of antibody-antigen complex in the biological fluid being positively correlated with the amount of anti-Neospora antibody in the biological fluid.

13. The immunological assay method of claim 12 wherein the Neospora spp is Neospora caninum.

14. The immunological assay method of claim 12 wherein the biological fluid is obtained from an animal selected from the group consisting of cattle, dogs, sheep, horses, goats, buffalo, deer, and humans.

15. The immunological assay method of claim 12 wherein the biological fluid is selected from the group consisting of whole blood, serum, other blood fractions, lymph, pus, colostrum, placental fluid, mucous discharge, milk, urine, saliva, sweat, lachrymous fluid, gastric fluid, stomach contents, digestive fluid, intestinal fluid, synovial fluid, ascitic fluid, peritoneal fluid, pericardial fluid, pleural fluid, cerebrospinal fluid, labyrinthine fluid and reproductive fluid.

16. The immunological assay method of claim 12 wherein the recombinant antigen is attached to a surface.

17. The immunological assay method of claim 12 wherein the detection of the antibody-antigen complexes comprises a secondary binding component selected from the group consisting of secondary antibody, Protein G, Protein A, antiIgG antiserum, antiIgG polyclonal antibody, antiIgG monoclonal antibody, antiIgG antibody-enriched fraction of serum, antiIgG antibody fragments, antiIgG recombinant antibody, and antiIgG recombinant antibody fragments.

18. The immunological assay method of claim 17 wherein the secondary binding component comprises a detectable marker component selected from the group consisting of an enzyme, a ligand, a biological receptor molecule, a fluorescent tag, a luminescent label, gold, and a radioactive label.

19. The immunological assay method of claim 18 wherein the detection of the amount of antibody-antigen complexes is selected from the group consisting of:
measuring a product formed or a substrate lost in an enzyme-catalyzed reaction, measuring binding in a receptor-ligand binding interaction; fluorometric determination of a fluorescent tag, photometric determination of a luminescent label, and radiometric determination of a radioactive label.

20. An ELISA assay for detecting anti-Neospora antibodies in a biological fluid, comprising the steps of:
a) providing the recombinant antigen of claim 8;
b) affixing the recombinant antigen to a surface to form a surface-bound antigen;
c) contacting the antigen with a biological fluid to be tested, under conditions wherein antibodies present in the biological fluid that are immunologically specific for the recombinant antigen bind to the antigen to form antibody-antigen complexes that are bound to the surface;
d) washing the surface to remove unbound antibody and other components of the biological fluid;
d) contacting the washed antibody-antigen complexes with a Protein G-horseradish peroxidase complex;
e) detecting the antibody-antigen complexes by performing an enzymatic assay of the horsradish peroxidase that forms a detectable product; and
f) measuring the amount of detected antibody-antigen complexes, the amount of antibody-antigen complexes in the biological fluid being positively correlated with the amount of anti-Neospora antibodies in the biological fluid.

21. A method of assessing a disease status of an animal with respect to the disease, neosporosis, comprising the following steps:
a) establishing at least one criterion of the disease status with respect to neosporosis, the criterion being based on a measure of anti-Neospora antibodies detected by the method of claim 12;
b) performing the method of claim 9 on the animal to determine a titer of the anti-Neospora antibodies in an isolated biological fluid from the animal;
c) correlating the determined titer with those of known positive and negative control samples;
d) optionally, further correlating the determined titer and known positive and negative control samples with a standard curve;
e) comparing the titer obtained with the at least one criterion for determinations of disease status; and,
f) assessing the disease status of the animal according to the at least one criterion.

22. A kit for the immunological detection of anti-Neospora antibodies comprising the recombinantly-expressed antigen of claim 8 and optionally, one or more of the following:
a) serum from a known infected animal for use as a positive serum control;
b) serum from an uninfected animal for use as a negative serum control;
c) a blocking agent;
d) one or more buffers;
e) a secondary binding component selected from the group consisting of protein G, protein A, and a secondary antibody immunologically-specific for antibodies of an animal species being tested;
f) reagents for a secondary detection system;
g) instructions for use;
h) one or more certificates of quality control; and
i) controls for a calorimetric detection system.


SEQUENCE LISTING (6 sequences)

SEQ ID NO: 1 - DNA, 888 bp, Neospora caninum
catatgtcagaaaaatcacctctacttgtcaatcaagttgtcacctgtgacaacgaagagaaatcatcagttgccgtcctactatcaccgaagctgaaccacatcacgctcaagtgccctgacaattcgaccgccgtgcccgctgctcttggttatccaacaaacaggaccgtctgcccggcggagtccggaggtcaaacttgtacaggcaaggagataccgttggaaagcctgcttcccggggcaaacgatagctggtggtcaggtgttgatatcaagactggcgttaagctcacaattcctgaagcgagcttccccacaacatccaagtcgttcgacgtcggctgcgtcagcagtgatgccagcaagagttgtatggtcacagtcacagtgccacccagagcctcatcgcttgtcaacggtgtcgcaatgtgctcttacggtgcaaacgaaactctcggccctatcacattgtccgagggcggatcttctacgatgaccctcgtttgcggcacggatgggaagccagttcctcctgatcctaagcaggtttgttctgggacgaccgtcaaggattgtaaagcaaaaccgttcactgatgttttcccaaaattcagtgctgattggtggcagggaaaacccgacactaaggatggtgcaaaactaacgatcaagaaaggtgcatttcctccaaaggaggaaaagtttactcttgggtgcaagagcgtatcgagtccggaggtttactgtactgtgcaggtggaggcagagcgcgcgagtgcagggatcaagtcgtcggctgaaaatgttggtcgcgtttcccttttcgctgtaacaattggactcgtaggctcgatagcgctcgagcaccaccaccaccaccactga

SEQ ID NO: 2 - DNA, 1138 bp, Neospora caninum
cttgtatcacaggtgcctttgtcgtacataaacattgtttcgattgtagtctagtcacaccgcactcgtttcatcactggcgcttttgtttattcatcgaatatgtttcctcgggcagtgagacgcgccgtctcggtgggtgtgttcgccgcgcccgcactggtggcgttctttgactgtggaactatggcatcagaaaaatcacctctacttgtcaatcaagttgtcacctgtgacaacgaagagaaatcatcagttgccgtcctactatcaccgaagctgaaccacatcacgctcaagtgccctgacaattcgaccgccgtgcccgctgctcttggttatccaacaaacaggaccgtctgcccggcggagtccggaggtcaaacttgtacaggcaaggagataccgttggaaagcctgcttcccggggcaaacgatagctggtggtcaggtgttgatatcaagactggcgttaagctcacaattcctgaagcgagcttccccacaacatccaagtcgttcgacgtcggctgcgtcagcagtgatgccagcaagagttgtatggtcacagtcacagtgccacccagagcctcatcgcttgtcaacggtgtcgcaatgtgctcttacggtgcaaacgaaactctcggccctatcacattgtccgagggcggatcttctacgatgaccctcgtttgcggcacggatgggaagccagttcctcctgatcctaagcaggtttgttctgggacgaccgtcaaggattgtaaagcaaaaccgttcactgatgttttcccaaaattcagtgctgattggtggcagggaaaacccgacactaaggatggtgcaaaactaacgatcaagaaaggtgcatttcctccaaaggaggaaaagtttactcttgggtgcaagagcgtatcgagtccggaggtttactgtactgtgcaggtggaggcagagcgcgcgagtgcagggatcaagtcgtcggctgaaaatgttggtcgcgtttcccttttcgctgtaacaattggactcgtaggctcgatagcggctggcgtcgcgtgagtgacaatcgttctgctcgccattcataaaaataatgcaagacatgttcgcgttcgtcatgtgtgtctttatcata

SEQ ID NO: 3 - protein, 294 aa, Neospora caninum
MSEKSPLLVNQVVTCDNEEKSSVAVLLSPKLNHITLKCPDNSTAVPAALGYPTNRTVCPAESGGQTCTGKEIPLESLLPGANDSWWSGVDIKTGVKLTIPEASFPTTSKSFDVGCVSSDASKSCMVTVTVPPRASSLVNGVAMCSYGANETLGPITLSEGGSSTMTLVCGTDGKPVPPDPKQVCSGTTVKDCKAKPFTDVFPKFSADWWQGKPDTKDGAKLTIKKGAFPPKEEKFTLGCKSVSSPEVYCTVQVEAERASAGIKSSAENVGRVSLFAVTIGLVGSIALEHHHHHH

SEQ ID NO: 4 - protein, 319 aa, Neospora caninum
MFPRAVRRAVSVGVFAAPALVAFFDCGTMASEKSPLLVNQVVTCDNEEKSSVAVLLSPKLNHITLKCPDNSTAVPAALGYPTNRTVCPAESGGQTCTGKEIPLESLLPGANDSWWSGVDIKTGVKLTIPEASFPTTSKSFDVGCVSSDASKSCMVTVTVPPRASSLVNGVAMCSYGANETLGPITLSEGGSSTMTLVCGTDGKPVPPDPKQVCSGTTVKDCKAKPFTDVFPKFSADWWQGKPDTKDGAKLTIKKGAFPPKEEKFTLGCKSVSSPEVYCTVQVEAERASAGIKSSAENVGRVSLFAVTIGLVGSIAAGVA

SEQ ID NO: 5 - DNA, 29 bp, Artificial Sequence
  synthetic sequence
gatccatatgtcagaaaaatcacctctac

SEQ ID NO: 6 - DNA, 26 bp, Artificial Sequence
  synthetic sequence
gatcctcgagcgctatcgagcctacg